(19) United States
(12) Patent Application Publication
(10) Pub. No.: US 2024/0164671 A1
(43) Pub. Date: May 23, 2024

(54) INTERACTIVE GAZE ANALYSIS KIT FOR THE EARLY DIAGNOSIS OF NEURODEGENERATIVE PATHOLOGIES

(71) Applicant: BIO AURUM S.R.L., Maglie (LE) (IT)

(72) Inventors: Maria Angela BAGNI, Maglie (LE) (IT); Silvia BISTI, Maglie (LE) (IT); Massimo CONTINI, Maglie (LE) (IT); Giacomo RONTINI, Maglie (LE) (IT)

(21) Appl. No.: 18/552,078

(22) PCT Filed: Mar. 25, 2022
(86) PCT No.: PCT/IB2022/052750

§ 371 Date: Sep. 22, 2023

(30) Foreign Application Priority Data
Mar. 25, 2021 (IT) 102021000007313

(51) Int. Cl. A61B 5/16; A61B 3/14; A61B 5/00

(52) U.S. Cl. CPC A61B 5/163; A61B 3/145; A61B 5/162; A61B 5/4082; A61B 5/7282; A61B 5/742; A61B 5/7475; A61B 2560/0443; A61B 2562/185

(57) ABSTRACT

Disclosed is an interactive gaze analysis kit for the early diagnosis of neurodegenerative pathologies essentially composed of a video camera with framing on the eyes of the patient and a screen both connected to a calculation unit through which the user is shown images specifically designed for being able to observe the presence of micronystagmus, the reaction times following an optical stimulations and measure the light sensitivity threshold, through which the diagnosis can be found. Since the test is particularly sensitive to the level of brightness to which the patient is subjected, it is necessary to have the presence of a dark tunnel through which the patient can look at the screen, an infrared, visible spectrum or infrared and visible spectrum lighting system, an image analysis software and input-output peripheral devices for allowing the interaction both with the doctor and with the patient. Other related embodiments are described.

### FIELD OF THE ART

The present invention operates in the field of systems for the diagnosis of neurodegenerative pathologies and, in particular, is based on eye-tracking technology and, more particularly, on the analysis of gaze movements and latencies.

### PRIOR ART

Neurodegenerative diseases such as Alzheimer's, Parkinson's, multiple sclerosis etc. have, from the clinical standpoint, a variety of symptoms that characterize the different pathologies and the progression of the degenerative processes.

Among the symptoms taken under consideration, the visual function is often overlooked. The retina, nervous component of the eye bulb is effectively part of the central nervous system and cannot be extraneous to the above-indicated degenerative processes. Experimental results, especially in animal models and observations in patients, have confirmed this hypothesis.

Recently, there has been increased attention on the visual function and on the deterioration thereof for two reasons: firstly, since sight is fundamental for maintaining a good quality of life in patients, particularly in Parkinson's and in sclerosis where the motor activity is compromised and a correct time-space evaluation of the surrounding environment can be fundamental for preventing collateral damage such as falls or other. Secondly, since the access to the visual function is relatively simple and it could become an early diagnostic system for the pathological process, and be practical for monitoring the effectiveness of therapeutic treatments.

In order to reach these two objectives in the present invention, a system is described for carrying out a simple diagnostic test, capable of measuring, in a quantitative manner, perceptive thresholds, latencies, response amplitudes and easily measurable functional deficits, like visual-motor coordination (saccades tied to perceptive thresholds), easy-to-use even for the general practitioner and, above all, low cost for a widespread use through the territory. For this purpose, the system described hereinbelow is capable both of generating visual stimuli that is quantitatively characterized, and of recording the visual and motor responses in an accurate manner.

At the present state of the art, there are many devices that carry out eye movement analysis, usually intended for a generic use, which must therefore be integrated in an ad hoc system in order to perform the requested functions. In the medical field, the most frequent applications regard the use for surgical purposes or the use for treating the visual apparatus itself.

The objective of the present invention is instead that of creating a device with a very well identified purpose, dedicated for a very specific task, for which the devices present on the market have characteristics that are not entirely adherent to the needs set forth hereinbelow. In the patent CA2827498A1, a method is published for the differential diagnosis between Parkinson's disease and progressive supranuclear paralysis. Nevertheless, the patent explicitly states that any one eye-tracking system is usable for this type of application, recommending a sampling frequency of the movement of at least 500 Hz. In order to be able to extend the diagnosis to other types of degenerative diseases, it is however necessary to account for the sensitivity to brightness, a problem not confronted in this patent but brilliantly resolved in the invention described hereinbelow.

The attention to the brightness is not even considered in the patents US2016106315A1 and US2014221869A1, in which eye-tracking solutions are proposed for the diagnosis of neurological diseases, which are nevertheless simplified and do not account for the interference of the environmental light in the precision of the diagnosis of specific pathologies, which can instead be covered due to the present invention.

There is no doubt, therefore, that such solutions only partly confront the critical items described above.

### DESCRIPTION OF THE INVENTION

According to the present invention, an interactive gaze analysis kit is attained for the early diagnosis of neurodegenerative pathologies which effectively resolves the abovementioned problems. The central nervous system and the eye are deeply connected both functionally and physiologically. The bond between these two structures starts during the embryonic formation, when the optical vesicle is formed from the embryonic layer assigned to form the nervous system. The visual pathways are constituted by photoreceptors which transduce the captured light impulse into nervous impulse that will then be sent from the nervous retinal cells, which constitute the optical nerve, to the visual cortex of the brain. Clinical and basic research show that there are neurodegenerative diseases and pathologies that affect the central nervous system which show particular alterations at the eye. For example, in Alzheimer's disease, characterized by the depletion of neurons at the cerebral cortex which determines alterations of the memory, dysfunctions of language and of perception, the patients affected by this pathology experience alterations of the visual field, reduction of the sensitivity to contrast, perception of movement, altered viewing of colors and alterations of the oculomotor function. Also in Parkinson's disease, caused by the loss of dopaminergic neurons of the brainstem, there is a clear effect on the vision. Indeed, dopamine is an important neurotransmitter even at the retina, dopaminergic cells are present in the inner plexiform of the eye and receptors for dopamine were identified on the cells of the retinal pigment epithelium, on the photoreceptors, and on the ganglia cells. The dopamine modulates the receptor fields of the retinal cells in order to ensure sensitivity to contrast, vision of the colors and it has a role in adaptation to light. At the eye, the patients affected by Parkinson's disease can experience defects of convergence and reduction of visual acuity. Multiple sclerosis, another neurodegenerative disease, is characterized by a demyelination of the axons. Optic neuritis is the first clinical manifestation of the disease in about 20% of the patients and 75% of the patients affected by the sclerosis have had at least one episode during their lifetime. The visual symptoms are the result of the demyelination of the visual pathways and the consequent optic neuritis appears with monolateral reduction of the visual acuity, of the color vision and of the sensitivity to contrast and with alteration of the visual field. The existence of these eye alterations, in many neurodegenerative pathologies, confirmed in various studies and various authors, underlines the strong bond between the eye and Central Nervous System (CNS). In many cases, the eye manifestations precede the central symptoms, suggesting that the eye imaging can be a valid aid in the early diagnosis of these diseases. From many aspects, the eye is a privileged site for research since the eye structures are easily accessible from outside and it is possible to examine them in vivo without any risk for the patient. Presently there are many diagnostic devices which analyze the eye movement and, generally, visual perception, but most of these are limited to the functional analysis of the eye, not including a diagnostic connection with other pathologies of neurodegenerative type.

The present invention instead has the object of creating a device which allows, through an integrated analysis of the visual perception, monitoring and measuring a series of parameters, an early diagnosis of several neurodegenerative diseases, like those mentioned above. In order to effectively describe the characteristics of the device of the present patent, it is useful to underline the difference between eye tracking and gaze analysis, the first is involved with measuring the eye rotations with respect to a reference, such as the head of the subject, while the second analyzes the point actually looked at within the scene. While the diagnostic systems presently on the market are directed towards gaze tracking, the present system instead focuses on the analysis of the eye movement of the patient, while he/she is asked to look at a specific point in space and at the same time stimulated by means of specific and localized light stresses. With this simplification, one knows ahead of time the direction and the distance of the point to be observed and hence it is not necessary to calculate it; indeed the direction of the gaze is not the first objected to be attained, but in the end the information of interest is contained in the capacity of the eye movement of the patient when it is stressed by light stimuli or the point to be observed is moved. The interactive present system of gaze analysis carries out an analysis and a monitoring of the eye movement of a patient by underlining, by means of a compared analysis and a calculation of pre-established parameters, the presence of behaviors of the visual apparatus which signal the possible presence of neurological pathologies. The system is constituted by a video camera which carries out a filming with a spatial and temporal resolution of the eyes of the patient sufficient for meeting the relative accuracy requirements. The camera, for example comprising a charge-coupled device (CCD), may be suitable for use in the visible spectrum, or with infrared wavelengths of light, or with visible spectrum and infrared wavelengths. The filming is carried out in complete darkness and while the patient directs his/her gaze towards a suitable screen i.e. display which shows the test images which stimulate the eye movement. For the diagnostic test to which the patient is subjected, it is necessary to have an appropriate screen e.g., an LCD screen e.g. with LED backlighting. A further linear polarizing filter may optionally be added, in frontal position, at the end of the light path. Ideally the luminance of the screen should be as low as possible. Such further linear polarizing filter modulates the quantity of light exiting from the screen, thus to modify the luminance thereof, lowering it, so as to eliminate all the reflections from the image of the eyes which is registered by the video camera. The polarized filter can be held fixed or rotated by means of an electronic actuator connected to the software equipping the present system, so to analogically modify the effect. In order to ensure a complete darkness during the analysis, the present system is provided with a dark tunnel or box which prevents the passage i.e ingress of external light. The patient looks at the screen through the tunnel or box, e.g. made of fabric or rigid material with an opening at the patient side end so as to allow him/her to position the face with the gaze directed towards the interior of the tunnel or box, in any case without penetrating the external opening. The patient side of the screen, integral with the vision system and with the dark tunnel, is mounted on a vertical axis translating in a manner such that it is possible to adjust the height of the abutment and be adapted to the various situations of patients and abutment planes with different heights. This end, in order to ensure health safety, can also have an automatic sterilization system based on emission of UV rays. In the tunnel embodiment, on the side opposite the opening for the face of the patient, another opening is made that is suitable for housing the screen directed towards the face of the patient. In the box embodiment, the box comprises (i.e. contains in its interior) the screen directed towards the face of the patient. Since the patient will be requested to operate in conditions of darkness, in order to illuminate the scene (i.e. particularly the face and especially the eyes of the patient), necessary for the filming of the video camera, an infrared lighting system will be employed e.g. by means of a system of one or more infrared LED illuminators with an emission frequency such that they are not visible; and optionally a filter for screening the visible light, thus ensuring the best performance. In an alternative embodiment, the lighting system is able to illuminate in infrared and the visible spectrum, for example via one or more types of illuminator for example covering all or parts of the infrared and visible spectral range. With such an arrangement, a patient may be first tested using visible spectrum illumination to acquire a first degree of information about the patient, for example the detection of some delay in ocular motor responses. Patients exhibiting such responses can then be subjected to a second level of analysis using infrared illumination to further characterize the deficit of the patient. In this embodiment, the video camera may optionally have a filter that is adapted to screen (i.e. remove) the visible light when infrared illumination is used. When a test is performed using visible spectrum illumination, optionally a range of luminance simulating the range from dusk until dawn is used. The position of the patient is provided with an input peripheral device e.g. comprising two buttons placed laterally with respect to the structure of the tunnel or box, one per hand. All the tests were designed for being executed with this extremely simple interface, such that they can be performed even by elderly patients. The device guides the patient in the test with instructions on the video and audio messages that ask the patient to respond, for example, to simple questions on the images shown by indicating, by means of the buttons, the selection that best approaches one's own visual perception. The physician, however, interacts with the device by means of a touchscreen display and a physical keyboard that allows the management of the tests and the display of the results and of the movement of the eyes of the patient to be examined, recorded by the video camera in real time. The device provides for various analysis modes, such as for example the display of micronystagmus, i.e. the display of a single image of a few strongly contrasted pixels and the amplitude and speed measurement during their fixing; or the analysis of the reflected saccades, i.e. the display of a small image with an instantaneous movement for measuring the amplitudes and the speeds with which a patient reacts. Once the data regarding the functioning of the visual apparatus has been acquired, due to the filming and recording of the video camera, this may be inserted in a calculation system that will perform all the computation functions regarding the collection and the analysis of the observed data. The software with which the present device is equipped is suitably able to process the set of input data such to detect the presence of micronystagmus, the reaction times following optical stimulations and to measure the light sensitivity threshold, which overall, subject to a specialist medical anamnesis, can allow an early diagnosis of a plurality of neurodegenerative pathologies. It is also possible to configure the device in a manner such that it subjects the patient to automatically preset tests, by means of, for example, the use of earphones through which the user can listen to the pre-recorded instructions of the tests, thus allowing a single doctor to coordinate research on multiple patients simultaneously, by means of the use of multiple devices. The components of such device have reduced dimensions and are highly modular so to facilitate the use and transport. Materially the device may appear as a suitcase that allows two mechanical configurations: one for transport and one for use, and during the latter the two halves of the “suitcase” are open at 180° and being aligned form the abutment base of the device itself. Also, the power supply of all the components of the system may be designed so to facilitate the use of the device; indeed it may be constituted by a battery in a manner such that the apparatus can be used free of constraints tied to the fixed power grid. In order to make the performance of the test more comfortable, the present system can be provided with an additional instrumentation for the environments where the test is performed. Since the patient must complete the test in complete darkness, at its conclusion, when he/she moves his/her face away from the dark tunnel or box, the patient might be uncomfortable or even undergo a light shock when exposed to the external light. In order to overcome this drawback, a suffused red-light lighting system can be installed in the environment where the test is performed so as to not create an excessive difference between the night vision of the patient during the test and the vision of the surrounding environment at the end of the test.

The advantages offered by the present invention are evident in light of the description set forth up to now, and will be even clearer due to the enclosed figures and to the relative detailed description.

### DESCRIPTION OF THE FIGURES

The invention will be described hereinbelow in at least a preferred embodiment by way of a non-limiting example with the aid of the enclosed figures, in which:

FIG. 1 shows the main elements constituting said system of gaze analysis for the purpose of early diagnosis of neurodegenerative pathologies. Said elements comprise: a video camera 100 adapted to record, with detail, every single movement of the eye of the patient, a LCD screen with LED backlighting 101 integrated with a linear polarizing filter 102 and a dark tunnel 103 adapted to ensure a complete darkness, preventing the passage i.e ingress of external light with an opening for the face that is height-adjustable 104 and which possesses an automatic sterilization system 600 and an input opening 105 adapted to house said screen 106 directed towards the face of the patient. The doctor interface also comprises a display 113 and at least an input peripheral device 114 dedicated to use by the medical personnel adapted to allow a constant monitoring. While the patient interface is characterized by an input peripheral device 111 comprising at least two buttons 112 adapted to allow a manual indication of one of the options proposed by the test, to which such patient is subjected. Said device is also provided with a double system: an infrared, visible spectrum, or infrared and visible spectrum lighting system 107, adapted to illuminate the scene for the filming by means of said video camera 100, provided with an optional filter 109 and a calculation system 110 adapted to perform all the computation functions regarding the collection and the analysis of the observed data, and which allows, through an electronic actuator 800, controlling said polarizing filter 102.

FIG. 2 instead illustrates several integrating devices which allow implementing the efficiency of the system. The figure shows a pair of earphones 200 adapted to allow an interaction with the automated user. In addition, a battery 400 is represented which is adapted to ensure the power supply of all the components of the system so as to be able to use the device when moving or generally free of direct electrical power supply constraints. Finally, so as to ensure an efficient management, said input and output peripheral devices 111 are made by means of a touchscreen 500 for use by the health personnel. Finally, the system has an optional further additional instrumentation represented by a suffused red-light lighting system 700 adapted to allow vision even in nocturnal settings.

### DETAILED DESCRIPTION OF THE INVENTION

The present invention will now be illustrated as a non-limiting or non-binding example with reference to the figures, which illustrate several embodiments relative to the present inventive concept.

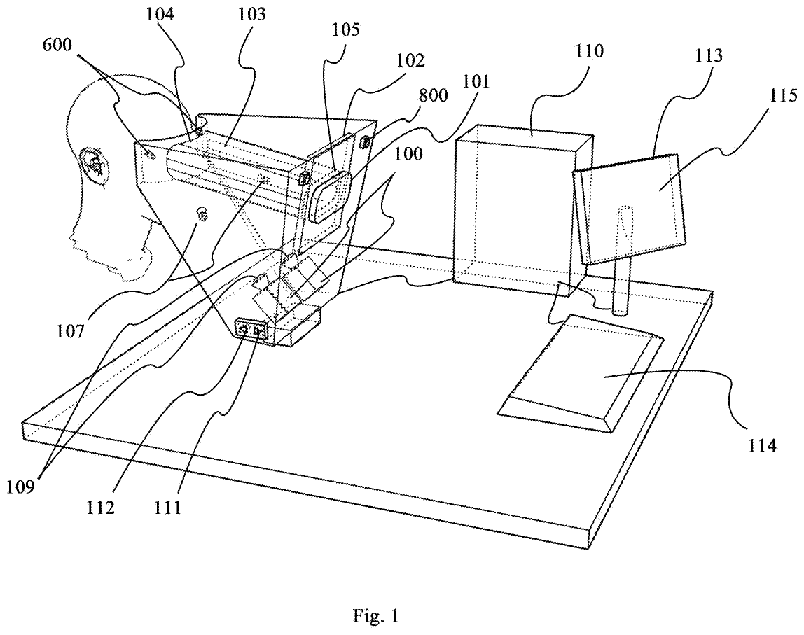

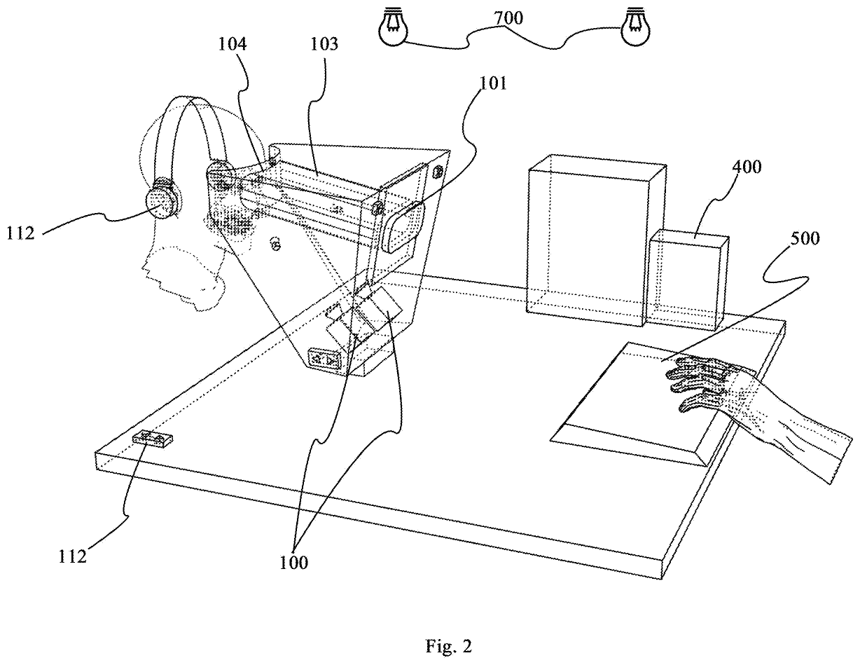

With reference to FIG. 1, said system, being adapted to allow an analysis and a monitoring of the eye movement of a patient, by calculating the presence of behaviors of the visual apparatus which signal the possible presence of neurological pathologies, comprises various components: a video camera 100 adapted to detect each eye movement through the monitoring of micronystagmus and saccades and reaction times following optical stimulations, a screen e.g. an LCD screen with LED backlighting 101 integrated with a linear polarizing filter 102, adapted to modulate the quantity of light exiting from said screen with very low luminance and a dark tunnel 103 adapted to ensure a complete darkness, preventing the passage i.e ingress of external light with an opening for the face 104 (adapted to allow the patient to position the face with the gaze directed towards the tunnel interior) which possesses an automatic sterilization system 600 by way of non-limiting example based on UV rays, and is height-adjustable, and an input opening 105 adapted to house said screen 106 directed towards the face of the patient. Instead of a dark tunnel, there may be provided a dark box having an opening for the patient's face and comprising the screen directed towards the face of the patient. With regard to the dedicated interface for the doctor, this may be characterized by a display 113 and at least an input peripheral device 114 dedicated to use by the medical personnel, adapted to allow a constant monitoring and an efficient management. Meanwhile, the patient interface may comprise an input peripheral device 111 constituted e.g. by at least two buttons 112 adapted to allow a manual indication of one of the options proposed by the test, to which said patient is subjected. The illustrated device is constituted by an infrared, visible spectrum, or infrared and visible spectrum lighting system 107, adapted to illuminate the scene for the filming by means of said video camera 100, optionally provided with filter 109 adapted to screen (i.e. remove) the visible light for example when only infrared illumination is used, and a calculation system 110 adapted to perform all the computation functions regarding the collection and the analysis of the observed data, and which allows, through an electronic actuator 800, controlling said polarizing filter 102. With reference to FIG. 2, on the other hand, several integrating devices of said system are represented, which are adapted to implement the efficiency thereof. Indeed, the figure shows earphones 200 adapted to allow an automated interaction with the user, through which the user can listen to pre-recorded instructions, allowing a single doctor to coordinate the activity of multiple devices simultaneously. The integrated system also provides for a battery 400 which power supplies all the components of the system such to be able to use the device when moving or, generally, free of direct electrical power supply constraints. In order to ensure an efficient management of the communication and interaction system, said input and output peripheral devices 111 are attained by means of a touchscreen 500 for use by the health personnel. Finally the image illustrates an additional instrumentation for the environments where the test is carried out, a suffused red-light lighting system 700, adapted to leave unaltered the night vision of the patient.

An interactive gaze analysis kit as described herein may be used for the early diagnosis of neurodegenerative pathologies and is adapted to allow an analysis and a monitoring of the eye movement of a patient, calculating the presence of behaviors of the visual apparatus which signal the possible presence of neurological pathologies.

Finally, it is clear that modifications, additions or variants that are obvious for a person skilled in the art can be made to the invention described up to now, without departing from the protective scope that is provided with the enclosed claims.

The invention may also be embodied by the following clauses:
1. An interactive gaze analysis kit (or device) for observing details of the functioning of a patient's visual apparatus, said system being characterized in that it comprises at least the following components:
   A) a video camera (100) adapted to frame the eyes of the patient and to record images, particularly images of the patient's eye movement, for subsequent processing;
   B) a screen (101) adapted to show the patient test images which stimulate the movements of the visual apparatus, said screen optionally integrated with at least a linear polarizing filter (102) adapted to modulate the quantity of light exiting from said screen wherein the screen with optional filter exhibits very low luminance;
   C) either i) a dark tunnel (103) adapted to ensure complete darkness, preventing the passage of external light; with an opening for the face (104) adapted to allow the patient to position his/her face with the gaze directed towards the tunnel interior, and with an input opening (105) adapted to house said screen (101) directed towards the face of the patient; or ii) a dark box adapted to ensure complete darkness, preventing the passage of external light; with an opening for the face (104) adapted to allow the patient to position his/her face with the gaze directed towards the box interior, wherein the box comprises said screen (101) directed towards the face of the patient;
   D) an infrared, visible spectrum, or infrared and visible spectrum lighting system (107), adapted to illuminate the scene for the filming by means of said video camera (100), optionally provided with filter (109) adapted to screen the visible light; and
   E) an input peripheral device dedicated to the patient (111) adapted to allow the user to respond to questions regarding the images shown;
   said system of components thus being adapted to allow observing details of the functioning of the patient's visual apparatus.
2. An interactive gaze analysis kit according to the preceding clause 1 characterized in that it further comprises feature F): a calculation system (110) adapted to perform all the computation functions regarding the collection and analysis of the observed data.
3. An interactive gaze analysis kit according to any one of the preceding clauses 1 or 2 characterized in that it further comprises feature G): a display (113) and at least an input peripheral device (114) dedicated for use by medical personnel, adapted to allow managing the test and displaying the results and the movement of the eyes of the user recorded by means of said video camera (100) in real time.
4. An interactive gaze analysis kit according to any one of the preceding clauses characterized in that it the screen (101) is integrated with at least a linear polarizing filter (102) adapted to modulate the quantity of light exiting from said screen.
5. An interactive gaze analysis kit according to any one of the preceding clauses characterized in that the screen (101) is an LCD screen with LED backlighting.
6. An interactive gaze analysis kit according to any one of the preceding clauses characterized in that it comprises a dark tunnel (103) adapted to ensure complete darkness, preventing the passage of external light; with an opening for the face (104) adapted to allow the patient to position his/her face with the gaze directed towards the tunnel interior, and with an input opening (105) adapted to house said screen (101) directed towards the face of the patient.
7. An interactive gaze analysis kit according to any one of the preceding clauses characterized in that it comprises an infrared, visible spectrum, or infrared and visible spectrum lighting system (107), adapted to illuminate the scene for the filming by means of said video camera (100), optionally provided with filter (109) adapted to screen the visible light.
8. An interactive gaze analysis kit according to any one of the preceding clauses characterized in that it allows an automated interaction with the user by means of the use of earphones (200) through which the user can hear pre-recorded instructions, allowing a single doctor to coordinate the activity of multiple devices simultaneously.
9. An interactive gaze analysis kit according to any one of the preceding clauses, characterized in that the described components have limited, modular size so as to allow an easy transportation of the apparatus.
10. An interactive gaze analysis kit according to any one of the preceding clauses, characterized in that the power supply of all the components of the system is attained by means of a battery (400) so as to be able to use the device when moving or generally free of direct electrical power supply constraints.
11. An interactive gaze analysis kit according to any one of the preceding clauses, characterized in that said input and output peripheral devices (111) are attained by means of a touchscreen (500) for use by health personnel.
12. An interactive gaze analysis kit according to any one of the preceding clauses, characterized in that said opening (104) for use by the patient possesses an automatic sterilization system (600), by way of a non-limiting example based on UV rays.
13. An interactive gaze analysis kit according to any one of the preceding clauses, characterized in that said opening (104) for use by the patient is height-adjustable.
14. An interactive gaze analysis kit according to any one of the preceding clauses, characterized in that it comprises, as additional instrumentation for the environments in which the test is carried out, a suffused red-light lighting system (700) adapted to leave unaltered the night vision of the patient.
15. An interactive gaze analysis kit according to any one of clauses 2 to 13, characterized in that said polarizing filter (102) can be controlled by means of said system of calculation (110), by means of an electronic actuator (800).
16. An interactive gaze analysis kit according to any one of the preceding clauses which, further by means of the aid of a software (115) of the system, is adapted to detect from the images recorded by said video camera (100) the presence of micronystagmus, the reaction times following optical stimulations and measure the light sensitivity threshold, which overall, subject to medical opinion, can allow an early diagnosis of a plurality of neurodegenerative pathologies.
17. An interactive gaze analysis kit according to any one of the preceding clauses characterized in that said lighting system (107) is a visible spectrum or an infrared and visible spectrum lighting system, and said video camera (100), is provided with a filter (109) adapted to screen visible light when infrared illumination is used.
18. Use of an interactive gaze analysis kit according to any one of the preceding clauses for the diagnosis of neurodegenerative pathologies.
19. Interactive gaze analysis kit for the early diagnosis of neurodegenerative pathologies, adapted to allow an analysis and a monitoring of the eye movement of a patient, calculating the presence of behaviors of the visual apparatus which signal the possible presence of neurological pathologies; said system being characterized in that it comprises at least the following components:
   A) a video camera (100) adapted to frame the eyes of the patient and to record the images for a subsequent processing;
   B) an LCD screen with LED backlighting (101) adapted to show the patient test images which stimulate the movements of the visual apparatus, indication of possible neurodegenerative pathologies and integrated with at least a linear polarizing filter (102) adapted to modulate the quantity of light exiting from said screen with very low luminance;
   C) a dark tunnel (103) adapted to ensure complete darkness, preventing the passage of external light; with an opening for the face (104) adapted to allow the patient to position his/her face with the gaze directed towards the tunnel interior, and with an input opening (105) adapted to house said screen (101) directed towards the face of the patient;
   D) an infrared lighting system (107), adapted to illuminate the scene for the filming by means of said video camera (100), provided with filter (109) adapted to screen the visible light;
   E) a calculation system (110) adapted to perform all the computation functions regarding the collection and analysis of the observed data;
   F) an input peripheral device dedicated to the patient (111) comprising at least two buttons (112) adapted to allow the user to respond to simple questions regarding the images shown, indicating the selection that is closest to his/her own visual perception; G) a display (113) and at least an input peripheral device (114) dedicated for use by the medical personal, adapted to allow managing the test and displaying the results and the movement of the eyes of the user recorded by means of said video camera (100) in real time;
   said system of components thus being adapted to allow observing details of the functioning of the patient's visual apparatus also by means of the aid of a software (115) of the system, adapted to detect from the images recorded by said video camera (100) the presence of micronystagmus, the reaction times following optical stimulations and measure the light sensitivity threshold, which overall—subjected to medical opinion—can allow an early diagnosis of a plurality of neurodegenerative pathologies.
20. Interactive gaze analysis kit for the early diagnosis of neurodegenerative pathologies, according to the preceding clause 19, characterized in that it allows an automated interaction with the user by means of the use of earphones (200) through which the user can hear pre-recorded instructions, allowing a single doctor to coordinate the activity of multiple devices simultaneously.
21. Interactive gaze analysis kit for the early diagnosis of neurodegenerative pathologies, according to any one of the preceding clauses 19 or 20, characterized in that the described components have limited, modular size so as to allow an easy transportation of the apparatus.
22. Interactive gaze analysis kit for the early diagnosis of neurodegenerative pathologies, according to any one of the preceding clauses 19 to 21, characterized in that the power supply of all the components of the system is attained by means of a battery (400) so as to be able to use the device when moving or generally free of direct electrical power supply constraints.
23. Interactive gaze analysis kit for the early diagnosis of neurodegenerative pathologies, according to any one of the preceding clauses 19 to 22, characterized in that said input and output peripheral devices (111) are attained by means of a touchscreen (500) for use by the health personnel.
24. Interactive gaze analysis kit for the early diagnosis of neurodegenerative pathologies, according to any one of the preceding clauses 19 to 23, characterized in that said opening (104) for use by the patient possesses an automatic sterilization system (600), by way of a nonlimiting example based on UV rays, and is also height-adjustable.
25. Interactive gaze analysis kit for the early diagnosis of neurodegenerative pathologies, according to any one of the preceding clauses 19 to 24, characterized in that it comprises, as additional instrumentation for the environments in which the test is carried out, a suffused red-light lighting system (700) adapted to leave unaltered the night vision of the patient.
26. Interactive gaze analysis kit for the early diagnosis of neurodegenerative pathologies, according to any one of the preceding clauses 19 to 25, characterized in that said polarizing filter (102) can be controlled by means of said system of calculation (110), by means of an electronic actuator (800).

1. An interactive gaze analysis kit for observing details of the functioning of a patient's visual apparatus, said system comprising:
A) a video camera adapted to frame the eyes of the patient and to record images for subsequent processing;
B) a screen adapted to show the patient test images which stimulate the movements of the visual apparatus;
C) either i) a dark tunnel adapted to ensure complete darkness, preventing the passage of external light; with an opening for the face adapted to allow the patient to position his/her face with the gaze directed towards the tunnel interior, and with an input opening adapted to house said screen directed towards the face of the patient; or ii) a dark box adapted to ensure complete darkness, preventing the passage of external light; with an opening for the face adapted to allow the patient to position his/her face with the gaze directed towards the box interior, wherein the box comprises said screen directed towards the face of the patient;
D) an infrared, visible spectrum, or infrared and visible spectrum lighting system, adapted to illuminate the scene for the filming by means of said video camera; and
E) an input peripheral device dedicated to the patient adapted to allow the user to respond to questions regarding the images shown;
said system of components thus being adapted to allow observing details of the functioning of the patient's visual apparatus.

2. The interactive gaze analysis kit according to claim 1, further comprising feature F): a calculation system adapted to perform all the computation functions regarding the collection and analysis of the observed data.

3. The interactive gaze analysis kit according to claim 1, further comprising feature G): a display and at least an input peripheral device dedicated for use by medical personnel, adapted to allow managing the test and displaying the results and the movement of the eyes of the user recorded by means of said video camera in real time.

4. The interactive gaze analysis kit according to claim 1, wherein the screen is integrated with at least a linear polarizing filter adapted to modulate the quantity of light exiting from said screen.

5. The interactive gaze analysis kit according to claim 1, wherein the screen is an LCD screen with LED backlighting.

6. The interactive gaze analysis kit according to claim 1, further comprising a dark tunnel adapted to ensure complete darkness, preventing the passage of external light; with an opening for the face adapted to allow the patient to position his/her face with the gaze directed towards the tunnel interior, and with an input opening adapted to house said screen directed towards the face of the patient.

7. The interactive gaze analysis kit according to claim 1, further comprising an infrared lighting system, adapted to illuminate the scene for the filming by means of said video camera, provided with filter adapted to screen the visible light.

8. The interactive gaze analysis kit according to claim 1, wherein the interactive gaze analysis kit allows an automated interaction with the user by means of the use of earphones through which the user can hear pre-recorded instructions, allowing a single doctor to coordinate the activity of multiple devices simultaneously.

9. The interactive gaze analysis kit according to claim 1, wherein the described components have limited, modular size so as to allow an easy transportation of the apparatus.

10. The interactive gaze analysis kit according to claim 1, wherein the power supply of all the components of the system is attained by means of a battery so as to be able to use the device when moving or generally free of direct electrical power supply constraints.

11. The interactive gaze analysis kit according to claim 1, wherein said input and output peripheral devices are attained by means of a touchscreen for use by health personnel.

12. The interactive gaze analysis kit according to claim 1, wherein said opening for use by the patient possesses an automatic sterilization system and is also height-adjustable.

13. The interactive gaze analysis kit according to claim 1, wherein said opening for use by the patient is height-adjustable.

14. The interactive gaze analysis kit according to claim 1, further comprising, as additional instrumentation for the environments in which the test is carried out, a suffused red-light lighting system adapted to leave unaltered the night vision of the patient.

15. The interactive gaze analysis kit according to claim 2, wherein said polarizing filter can be controlled by means of said system of calculation, by means of an electronic actuator.

16. The interactive gaze analysis kit according to claim 1 which, further by means of the aid of a software of the system, is adapted to detect from the images recorded by said video camera the presence of micronystagmus, the reaction times following optical stimulations and measure the light sensitivity threshold, which overall—subject to medical opinion—can allow an early diagnosis of a plurality of neurodegenerative pathologies.

17. The interactive gaze analysis kit according to claim 1, wherein said lighting system is a visible spectrum or an infrared and visible spectrum lighting system, and said video camera, is provided with a filter adapted to screen visible light when infrared illumination is used.

18. A method for the diagnosis of neurodegenerative pathologies, comprising providing the interactive gaze analysis kit of claim 1, and applying the kit to perform gaze analysis on a patient in need thereof.

19. The interactive gaze analysis kit according to claim 2, further comprising feature G): a display and at least an input peripheral device dedicated for use by medical personnel, adapted to allow managing the test and displaying the results and the movement of the eyes of the user recorded by means of said video camera in real time.

20. The interactive gaze analysis kit according to claim 19, wherein the screen is integrated with at least a linear polarizing filter adapted to modulate the quantity of light exiting from said screen.
